(19) United States
(12) Patent Application Publication
(10) Pub. No.: US 2025/0057451 A1
(43) Pub. Date: Feb. 20, 2025

(54) CALIBRATION METHOD AND SYSTEM

(71) Applicant: RSP SYSTEMS A/S, ODENSE S (DK)

(72) Inventors: Signe Maria LUNDSGAARD-NIELSEN, ODENSE S (DK); Anders PORS, ODENSE S (DK); Anders WEBER, ODENSE S (DK)

(21) Appl. No.: 18/712,567

(22) PCT Filed: Oct. 12, 2022
(86) PCT No.: PCT/EP2022/078431

§ 371 Date: May 22, 2024

(30) Foreign Application Priority Data
Nov. 23, 2021 (GB) 2116869.5

(51) Int. Cl. A61B 5/1495; A61B 5/145; A61B 5/1455; G01N 21/27; G01N 21/65

(52) U.S. Cl. CPC A61B 5/1495; A61B 5/14532; A61B 5/1455; G01N 21/274; G01N 21/65; A61B 2560/0238

(57) ABSTRACT

A system and method of calibrating a model used in a device for non-invasive in vivo measurement of an analyte concentration using Raman spectroscopy, the device including an optical source for providing an optical signal and an optical detector to receive a Raman scattered optical output, the method including: in response to the laser source being directed to a user's skin gathering Raman scattered radiation from the user's skin and based on that, calibrating the model based on reference data and the received Raman spectra over an extended period of days, such as 10 days or more.

The present invention relates to a calibration method and system.

Diabetes mellitus, in its different forms, is affecting an increasing number of individuals and placing undue strain on national health care budgets. Estimates (from 2021) state that 537 million people worldwide suffer from diabetes whilst this number is predicted to increase to 783 million by 2045.

For control of treatment the self-monitoring of blood glucose is used. In Type 1 diabetes patients dosing of insulin is frequently based on 4 to 6 blood glucose determinations per day. For invasive methods, lack of compliance remains an impediment to efficient therapy, and it has therefore been a long-term goal to develop truly non-invasive techniques to measure the blood glucose levels of diabetes patients

The current clinical trend favours indwelling electrochemical sensors that allow for continuous glucose monitoring. However, the associated skin puncture and open wound are still required, with associated discomfort for the user and increased risk of infection. These sensors also suffer from a biocompatibility issues that limit their lives to a few weeks.

For many decades it has been a goal to develop non-invasive techniques to measure the blood glucose levels of diabetes patients but practical solutions for general use have so far not been developed. The majority of approaches have been based on optical measurement of glucose in tissues such as the skin. Amongst these, spectroscopic techniques, such as fluorescence, absorbance and Raman have attracted considerable attention. Despite the fact that Raman scattering is a weak process and thus results in a poor signal, a number of factors render it an attractive option as a spectroscopic technique for measurement of glucose, and indeed other analytes, concentrations in the skin of a user. They include the high chemical specificity, minimal interference from tissue water content and only a modest fluorescence background render the technique one of the most promising candidates for non-invasive glucose monitoring.

Since the first feasibility study of measuring blood glucose with near-infrared Raman spectroscopy in 1997 several groups have substantiated the fundamental effectiveness of the technology by quantitative measurements of glucose levels in vivo. However, these reports may be considered proof-of-concept only in the sense that all measurements were performed in a controlled environment whilst the predictive capabilities of the calibration model were assessed by cross-validation only.

In earlier publications and patent applications, the current applicant has described the design and development of a table-top, confocal near-infrared Raman instrument for intermittent glucose determination. The instrument uses the principle of critical-depth Raman spectroscopy, where measurements are taken from interstitial fluid within a defined stratum of the skin. It is worth noting that in contrast to previous technology that also utilizes a confocal setup to probe in the living part of the skin, the work of the current applicant is the first of its kind to systematically study the relation between probing depth and prospective performance of the Raman-based glucometer, thus allowing definition of a critical depth from which the Raman signal should be acquired.

In the current applicant's International application number WO2011/83111 (granted in many jurisdictions) there is described a method and apparatus for non-invasive in vivo measurement by Raman spectroscopy of glucose present in interstitial fluid in skin. Amongst other aspects there is described apparatus for non-invasive in vivo measurement by Raman spectroscopy of glucose present in interstitial fluid in the skin of a subject, comprising a light source, optical components defining a light path from said light source to a measurement location, a light detection unit, optical components defining a return path for Raman scattered light from said measurement location to said light detection unit, and a skin engaging member having a distal surface for defining the position of said optical components defining the return path with respect to a surface of said skin in use, and wherein said optical components defining a return path for Raman scattered light selectively transmit to said light detection unit light scattered from near said measurement location such that at least 50% of Raman scattered light received at the light detection unit originates at depths from 60 to 400 μm beyond said distal surface of the skin engaging member.

In the current applicant's co-pending and granted patent portfolio a number of apparatuses are described for use in determining a blood glucose concentration using Raman spectroscopy.

Several parameters are preferably needed to meet user expectations in order to reach practical utility for a non-invasive glucose monitor (NIGM). These include for example, accuracy, cost, size, ease of use, calibration requirement and calibration stability.

Calibration stability has long been a unique selling point for the most successful continuous glucose monitors while the more conventional finger prick devices have sought to lower the cost per measurement. While it is expected that eventually NIGM technologies will be able to outperform all other technologies on cost per measurement since there is no need for consumables, this can only be practically realised if there are low requirements for calibration or none at all.

There is a desire to maintain the calibration of a device once an initial calibration process has been performed.

Calibration accuracy is determined based on a measurement of mean absolute relative difference (MARD). Although MARD has some limitations it is a widely used metric. As explained in the article entitled International Consensus on Use of Continuous Glucose Monitoring, in Diabetes Care of the American Diabetes Association, of December 2017, 40(12); 1631-1640, MARD is currently the most common metric used to assess the performance of Continuous Glucose Monitoring (CGM) systems. MARD is the average of the absolute relative error between all CGM values and matched reference values. A small percentage indicates that the CGM readings are close to the corresponding reference glucose values, whereas the larger the MARD percentage, the greater discrepancies between the CGM and reference glucose values.

In the current applicant's International application number WO2016/03448 (granted in many jurisdictions) there is a described a method of precalibration of a device for measuring glucose and other analyte concentration using Raman spectroscopy. As it explains, Raman spectroscopy is used to obtain molecular information in particular rotation and/or vibrational spectra which are often referred to as ‘fingerprints’ related to a specific molecule. Information related to rotational, vibrational and/or electronic states of molecules can therefore be used to analyze a sample comprising a number of unknown molecular components, thereby obtaining knowledge about the molecular components in the sample.

A method is disclosed for predicting whether a spectrum of Raman signals received transdermally in a confocal detector apparatus and having at least one component expected to have an intensity representing the concentration of a skin component at a point of origin of said Raman signals below the surface of the skin will accurately represent said concentration, which method comprises analysing features of said spectrum relating to skin components other than the skin component the concentration of which is to be measured and thereby determining whether the Raman signals originate primarily within the stratum corneum so that the spectrum will be less likely to represent said concentration accurately or originate primarily below the stratum corneum so that the spectrum will be more likely to represent said concentration accurately.

According to a first aspect of the present invention, there is provided a method of calibrating a model used in a device for non-invasive in vivo measurement of an analyte concentration using Raman spectroscopy, the device comprising:
an optical source for providing an optical signal and an optical detector to receive a Raman scattered optical output, the method comprising:
in response to the laser source being directed to a user's skin gathering Raman scattered radiation from the user's skin and based on that, calibrating the model based on reference data and the received Raman spectrum over an extended period of days; and, once calibrated after the extended period of days, storing the model for use.

A method is provided in which an extended calibration period is used and as an unexpected consequence calibration stability has been achieved. Typically, an extended period might be at least 10 days and preferably more than 2 weeks. More preferably, the extended period is more than 20 days. Preferably the extended period is between 20 and 30 days.

It has been recognised that useful calibration stability can only be achieved if sample variability is built into the model and since the stratum corneum turnover period and replacement of melanin greatly impacts the optical background signal, data from a full stratum corneum turnover period is likely needed in order to obtain a stable calibration. As demonstrated by the results and examples below an extended calibration time provides stability that is superior to that of a shorter calibration time of 4 days with a high number of measurements. Thus, in contrast to conventional systems in which it was understood that the number of data points in a calibration set was determinative for calibration stability, the present applicants have recognised that an important factor as well as individual tests or data points in the calibration is the period of calibration. By requiring an extended calibration period improvements in calibration stability are achieved.

In an embodiment, the calibration period is in excess of 10 days.

In an embodiment, the calibration period is in excess of 14 days.

In an embodiment, the calibration period is in excess of 20 days.

In an embodiment, the calibration period is between 20 and 28 days.

In an embodiment, the method comprises providing a model for calibrating and then modifying the model in dependence on calibration data received during the calibration period.

In an embodiment, the method comprises receiving reference data from an invasive blood glucose monitoring system or device.

In an embodiment, the method comprises releasing the calibrated model for use only if release criteria are satisfied.

According to a second aspect of the present invention, there is provided a system for calibrating a model used in a device for non-invasive in vivo measurement of an analyte concentration using Raman spectroscopy, the device comprising:
an optical source for providing an optical signal and an optical detector to receive a Raman scattered optical output, the system comprising:
a laser source to direct laser light to a user's skin
a detector to gather Raman scattered radiation from the user's skin and
a processor arranged, based on the received Raman scattered radiation and on reference data, to calibrate the model over an extended period of days and,
once calibrated after the extended period of days, to store the model for use.

In an embodiment, the calibration period is in excess of 10 days.

In an embodiment, the calibration period is in excess of 14 days.

In an embodiment, the calibration period is in excess of 20 days.

In an embodiment, the calibration period is between 20 and 28 days.

In an embodiment, the system comprises providing a model for calibrating and then modifying the model in dependence on calibration data received during the calibration period.

In an embodiment, the system comprises receiving reference data from an invasive blood glucose monitoring system or device.

In an embodiment, the system comprises releasing the calibrated model for use only if release criteria are satisfied.

Embodiments of the present invention will now be described in detail with reference to the accompanying drawings, in which:

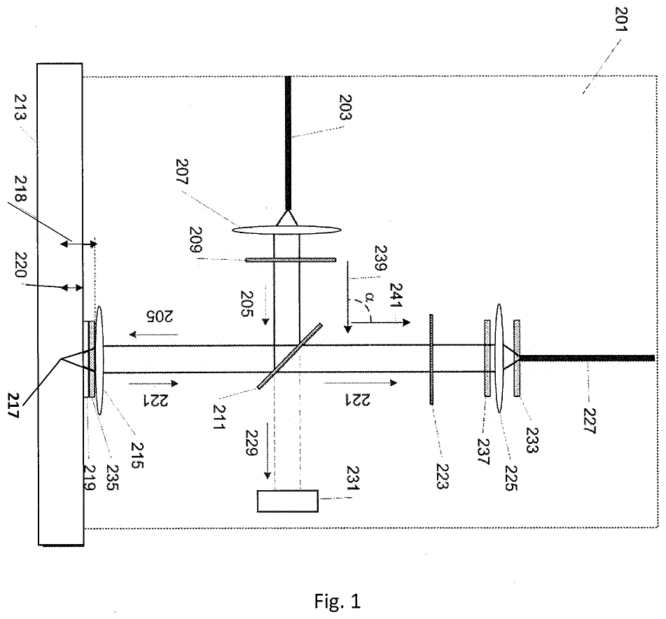

FIG. 1 is a schematic view of an optical configuration for taking non-invasive measurements of glucose concentration using Raman spectroscopy;

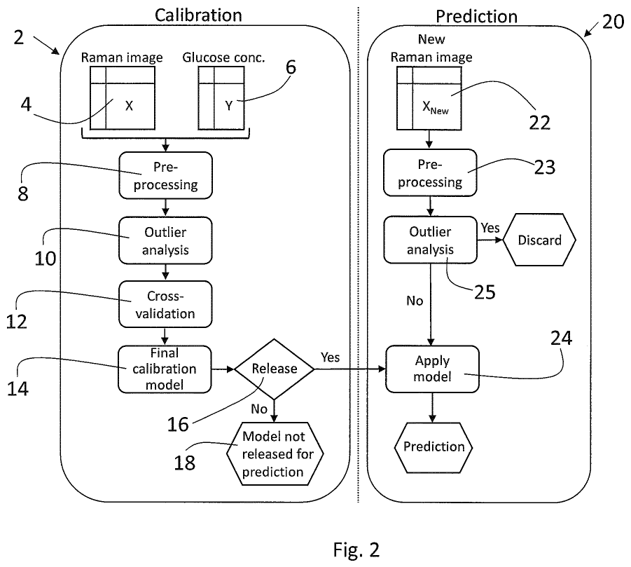

FIG. 2 is a flow chart showing a schematic view of a calibration and validation method;

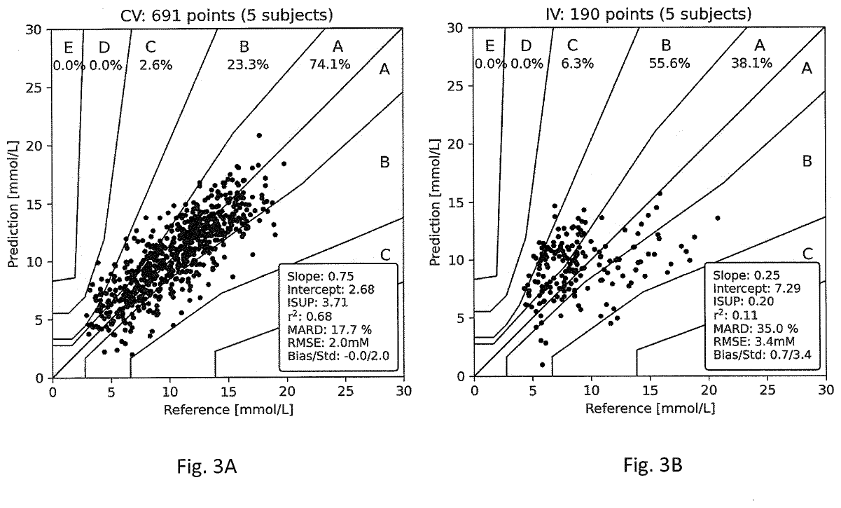

FIGS. 3A and 3B show respectively the cross-validation and independent validation results from a first calibration method;

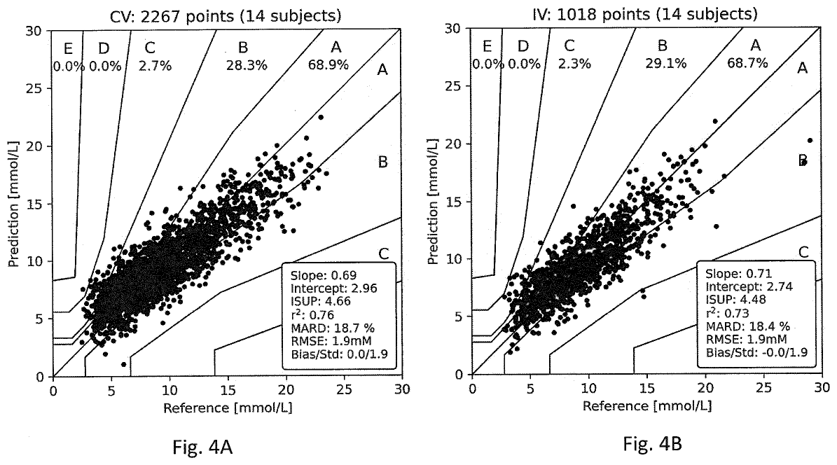

FIGS. 4A and 4B show respectively the cross-validation and independent validation results from a second calibration method;

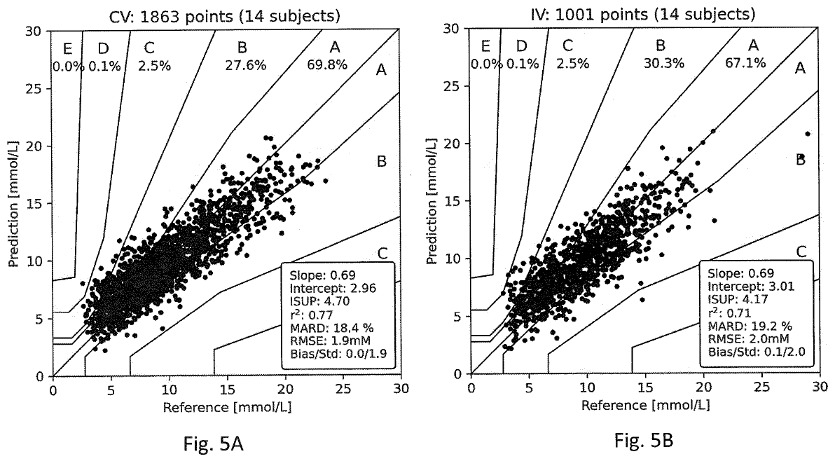

FIGS. 5A and 5B show respectively the cross-validation and independent validation results from a third calibration method;

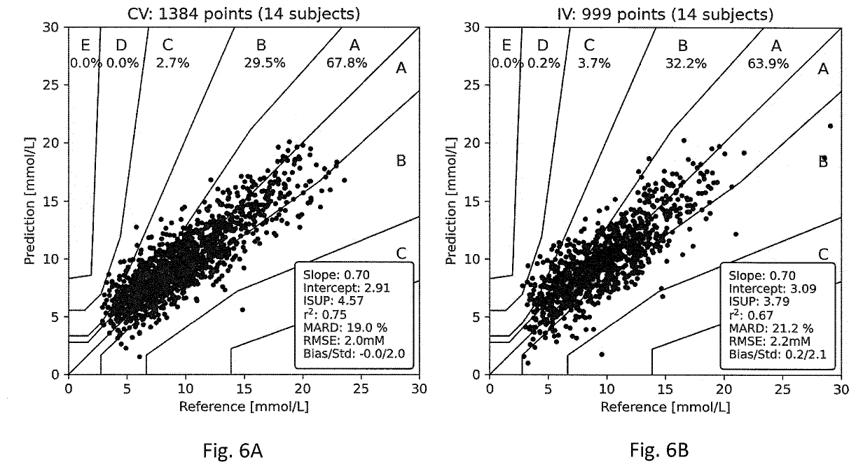

FIGS. 6A and 6B show respectively the cross-validation and independent validation results from a fourth calibration method;

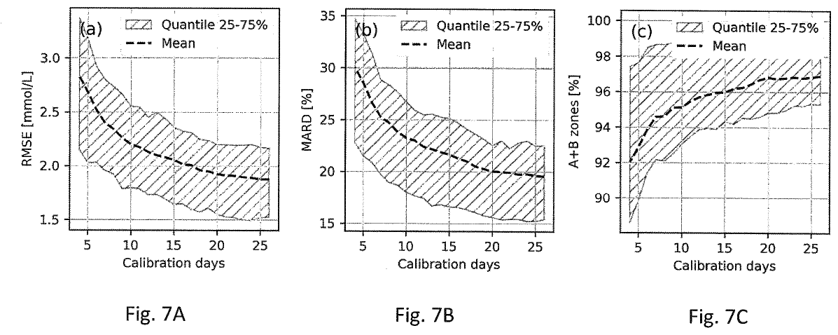

FIGS. 7a to 7c, show respectively examples of variation of RMSE, MARD and percentage of A+B zones in CEGs, with days of calibration; and

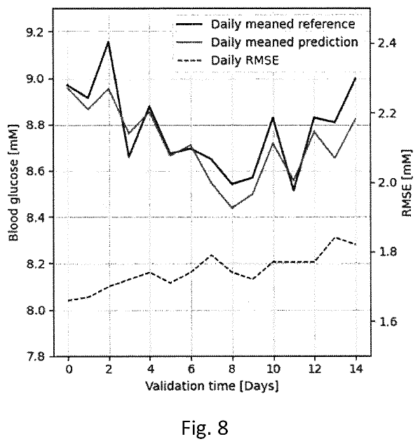

FIG. 8 shows a comparison between the daily mean of the predicted and reference glucose value and RMSE for included patients for a validation period of 15 days.

FIG. 1 is a schematic view of a non-limiting example of an optical configuration for taking non-invasive measurements of glucose concentration using Raman spectroscopy.

The basis for a spectroscopic setup is a light source, e.g. a laser, which is used for illuminating a sample. The light from the light source (the incoming light) will interact with the sample, and often result in an alternation of the light which is transmitted through, emitted by, reflected by and/or scattered by the sample. By collecting the altered light and analyzing its spectral distribution, information about the interaction between the incoming light and the molecular sample can be obtained; hence information about the molecular components can be obtained.

The spectral distribution is typically measured by using a spectrometer. A spectrometer is an optical apparatus that works by separating the light beam directed into the optical apparatus into different frequency components and subsequently measuring the intensity of these components by using e.g. a CCD detector, a CCD array, photodiode or such.

FIG. 1 shows a first embodiment of an optical configuration that might be included within an optical probe 201. The probe comprises a first optical fibre 203 for guiding light into the optical probe 201. The light source is normally a laser, and the optical configuration is shown merely as an example of a suitable optical configuration for use with the calibration method described herein.

Upon exiting the first fibre 203, the incoming light 205 is collimated using a first lens 207 and optically filtered by passing through a first filter 209 blocking any percentage between 0 and 100 of frequencies/wavelengths outside the laser frequency/wavelength. Blocking of frequencies outside the laser frequency ensures that e.g. fluorescence generated inside the first fibre 203 is removed from the incoming light 205. The first filter 209 may also block any percentage between 0 and 100 of the laser frequency. This is an advantage if the intensity of the incoming light 205 is too high for the requirements of the sample. The first filter 209 is preferably a band-pass filter, a notch filter, an edge filter or such.

The optical probe 201 further comprises a dichroic mirror 211 that either reflects or transmits any percentage between 0 and 100 of the light, where the percentage of reflected and transmitted light is dependent on the coating on the dichroic mirror 211, the angle at which the light hits the dichroic mirror 211, and the frequency of the light. The dichroic mirror 211 can e.g. be coated such that it reflects the highest percent of the incoming light 205 when the dichroic mirror 211 is positioned at a given angle in relation to the direction of the incoming light 205. Changing the angle between the dichroic mirror 211 and the incoming light 205 will therefore reduce the percent of incoming light 205 reflected by the dichroic mirror 211.

In this example, most of the incoming light 205 is reflected by the dichroic mirror 211 and focused inside the skin 213 of a subject by a second lens 215. The focus point 217 of the incoming light 205 is defined by the focal length 218 of the second lens 215 and the distance distal of the lens of a window 219 and in particular its distal surface which engages the skin in use. The second lens 215 is preferably convex, but could also be aspheric or planar.

The process of calibration and validation as it might be used within a NIGM will now be described with reference to FIG. 2.

FIG. 2 shows a simplified and schematic flow diagram of the steps in calibration and validation for a NIGM. Some of the steps could be omitted or indeed further steps included but the model shown is to represent a general and schematic process by which calibration may be performed.

The process can be thought of as including a calibration stage 2 in which a calibration model is produced and, once that is complete, if deemed acceptable according to some defined criteria, the calibrated model is released and can then be used in a prediction stage 20 when an actual result is required by a user. As the input to the calibration stage 2, a Raman image or spectrum 4, e.g., obtained from a spectrometer described above with reference to FIG. 1, is received together with reference data 6.

Data is pre-processed 8 to reduce noise and to normalize data, while spectral and reference outliers are removed in an outlier analysis 10. The remaining calibration data is utilized in a cross-validation analysis 12, which is simultaneously used to fit model parameters and estimate model performance. As the last step in the calibration stage 2, a final calibration model is constructed 14 by utilizing model parameters from the cross-validation 12, and, if the model fulfills pre-specified performance requirements, it is released 16. Alternatively, if the performance of the final model does not fulfil the specified performance requirements, i.e. its performance against those criteria or requirements is not good enough, it is not released 18 and cannot therefore subsequently be used to predict glucose concentration from Raman spectral measurements.

Looking at the prediction stage 20, a new Raman image or spectrum 22 is received from a user. Typically this is derived from use of a probe or apparatus such as that described above with reference to FIG. 1. The Raman spectrum 22 is, like in the calibration stage 2, preprocessed 23 and evaluated 25 for outliers. If recognized as an outlier by the outlier analysis 25, the measurement 22 is discarded and no prediction is made. On the other hand, if the measurement 22 passes the outlier analysis 25, the calibration model from 14 is applied 24, thereby resulting in a prediction of the glucose level.

The Raman images 22 that are input to the prediction stage 20 represent independent measurements and can be used for independent validation of the accuracy. It is clear, that the independent measurements made using the produced model from the calibration stage 2 provide a valid data set to evaluate the accuracy of the device and the calibration model produced by the calibration stage 2. Several examples will now be described in which subject-specific calibration models are produced and released and then independently validated as described above. The data is shown (in FIGS. 3 to 6) in the form of consensus error grids. A consensus error grid is a well-known tool for evaluating the accuracy of blood glucose meters. It comprises a 2 dimensional plot of measured blood glucose levels against the actual blood glucose level determined by a control method.

FIGS. 3A and 3B show respectively consensus error grids showing the data from the cross-validation and independent validation results from a first calibration method not according to the present invention. In this calibration method, a short calibration period of 4 days was used with a high number of measurements or samples taken in this period. As will be understood by a comparison of FIGS. 3A and 3B, the independent validation of the models, corresponding to the 5^{th} day of measurements, demonstrates a significantly higher MARD of 35.0% as compared to the MARD of 17.7% derived from the cross-validation. Collectively only 93.7% of the independent validation data is in zones A and B, compared to 97.4% in the cross-validated data.

The released models validated using this method are the models referred to herein as RSP12. The data set comprises 5 subjects with diabetes mellitus, and the paired data points in FIG. 3 are the outcome of the corresponding 5 calibration models, each model constructed with 4 days of measurements. The cross-validation data of FIG. 3A was derived using Leave One Out cross-validation. The reference data for both the cross validation and independent validation was derived using a Freestyle Libre CGM (Abbott, Illinois, USA). The cross-validated data shown in FIG. 3A demonstrates a good correlation between the non-invasive measurements and the reference concentrations.

It is noted in general that for the examples described herein, the reference concentration (comparison) measurements, used to compare the NIGM data during home session days, were performed using the Contour® next ONE (Ascensia Diabetes Care GmbH, Basel, Switzerland) blood glucose monitoring system (BGMS) in RSP19, RSP21, and RSP24 protocols and free style libre CGM in RSP12. Both systems are among the most accurate for practical home use.

For taking a measurement using an NIGM, a device similar to that shown schematically and described above with reference to FIG. 1 was used. Typically, a measurement process comprised two measurements with a total measurement time of 3 minutes within which multiple spectra were recorded. The integration time was subject-dependent and set to properly use the dynamic range of the image sensor.

With an output power of approximately 250 mW, integration times of a single measurement scan varied between 1.5-2.5 seconds. The spectral range is 300-1615 cm⁻¹, and within this range the average resolution is approximately 8 cm⁻¹. In the specific example used, the NIGM device uses a laser wavelength of 830 nm.

Referring to FIGS. 4A and 4B, consensus error grid plots are shown for a method according to the present invention. The protocols used, and in respect of which the data was collected, were known as RSP19 and RSP21.

A calibration period of 26 days was used in which the calibration data shown in FIG. 4A was collected. The determined MARD for the calibration data is 18.7% and the Zone A and B data accounts for 97.2% of the data.

FIG. 4B showed the independent validation data collected over a period of 14 days, with 5-6 measurements per day and per subject. FIG. 4 comprises data from 14 subjects and, hence, 14 calibration models. As can be seen, the MARD of the independent validation data is 18.4% and Zone A and B data accounts for 97.8% of the data. Accordingly, there is no deterioration in the independently validated data as compared to the cross-validation shown in FIG. 4A. By use of an extended calibration period, i.e. 26 days, calibration stability is thus achieved and empirically demonstrated. The calibrated model determined after the 26 day calibration period may be stored for use by a user and used subsequently to produce readings of blood glucose concentration based on capture of a Raman spectrum. Typically, a system for performing the method will include a processor and memory. The calibrated model is typically stored in the system memory for use.

A second example is shown with respect to FIGS. 5A and 5B. In this case, a calibration period of 20 days was used, whereas the validation data was identical to that of FIG. 4B. With a calibration period of 20 days the change in MARD from the calibration data to the independently validated data is again small. In this example, the MARD for the cross-validated data is 18.4% (Zones A and B 97.4%) whereas for the independent validation data derived from the models released based on the cross-validation data, a MARD of 19.2% is achieved (Zones A and B 97.4%). The calibrated model determined after the 20-day calibration period may be stored for use by a user and used subsequently to produce readings of blood glucose concentration based on capture of a Raman spectrum.

A third example is shown with respect to FIGS. 6A and 6B. In this example, the calibration period was 14 days, while the validation data (also extending a period of 14 days) remained identical to that of FIGS. 4B and 5B. In this case, it is again possible to achieve calibration stability, as demonstrated by the small change in performance metrics between cross-validated and independently validated data. For example, the MARD changes from 19.0% (Zones A and B 97.3%) in cross-validation to 21.2% (Zones A and B 96.1%) in independent validation.

Thus, it is demonstrated that for a Raman based non-invasive glucose monitoring device, it is possible to maintain calibration stability over an extended period of time. In respect of the RSP19 and RSP21, models upon which FIGS. 4, 5 and 6 (A and B) are based, the study time was 40 days and the full calibration period consisted of 1 in-clinic day and 25 days of out-patient measurements with a minimum of 6 measurements per day in the out-patient days and up to 32 measurements in the in-clinic day. The validation period consisted of 14 out-patient days. Measurements were distributed throughout the day, but subjects were asked not to perform measurements within 15 minutes of a meal and in addition, each measurement was requested to be at least one hour apart. This was done to secure as much variation as possible as a low amount of variation would limit the ability to properly calibrate and assess the correlation.

The disclosed method and control system thus demonstrates that a high level of calibration stability can be achieved based on several weeks of measurements. Importantly, in some cases portable devices are used such that there are no restrictions as to the subjects whereabouts whilst measuring.

The data from the FIGS. 3-6 are included in the table (Table 1) below.

TABLE 1

| | Avg. | | | | | | | | |
|---|---|---|---|---|---|---|---|---|---|
| | number of | | | | | | | | |
| | calibration | | | | | | | | |
| | points per | Days of | Days of | MARD | MARD | Delta | R² | R² | Delta |
| Protocol | model | calibration | validation | CV | IV | MARD | CV | IV | R² |
| RSP-12 | 138 | 4 | 1 | 17.7% | 35.0% | 17.3% | 0.68 | 0.11 | −0.57 |
| RSP19/21 | 162 | 26 | 14 | 18.7% | 18.4% | −0.3% | 0.76 | 0.73 | −0.03 |
| RSP19/21 | 133 | 20 | 14 | 18.4% | 19.2% | 0.8% | 0.77 | 0.71 | −0.06 |
| RSP19/21 | 99 | 14 | 14 | 19.0% | 21.2% | 2.2% | 0.75 | 0.67 | −0.08 |
| RSP24 | 141 | 26 | 15 | 16.6 | 19.0 | 2.4% | 0.78 | 0.71 | −0.07 |

As can be seen, where an extended period, i.e., more than, say, 10 or 14 days, of calibration is used, a high level of MARD consistency is achieved.

FIGS. 7a to 7c, respectively show evidence of variation of RMSE, MARD and percentage of A+B zones in CEGs, with days of calibration. As can be seen with a calibration period in excess of 10 days, achievement of low RMSE and MARD and high A+B zone percentage is possible. The data corresponds to protocol RSP24 included above in Table 1 for a calibration period of 26 days.

The data supporting the evidence in FIGS. 7a to 7c was based on a sample of 160 patients, encompassing 137 and 23 patients with type 1 or type 2 diabetes, respectively.

The patients with either type 1 or type 2 were recruited. Exclusion criteria were applied to select the participants in the study. Any suitable criteria could be used but in this case, the criteria included:
severe hypoglycaemia in the past 3 months
hypoglycaemia unawareness
pregnancy or lactation period
known severe allergy to medical-grade adhesive or isopropyl alcohol (used to clean the skin)
inability to comply with the study procedures (due to, e.g., known psychiatric diagnoses, lack of cognitive ability, alcohol dependency, drug use, or psychosocial overload)
severe diabetes-related complications (e.g., advanced autonomic neuropathy, kidney disease, foot ulcers, legal blindness, or symptomatic cardiovascular disease as evidenced by a history of cardiovascular episode(s))
systemic or topical administration of glucocorticoids for the past 7 days
inability to hold arm or hand still (including tremors and Parkinson's disease;
Extensive skin changes, tattoos or diseases on right thenar; or undergoing dialysis treatment.

All patients were screened with a skin tone sensor for skin type I to V according to the Fitzpatrick scale. Any suitable skin tone sensor could be used. In one example the skin tone sensor DEESS Demi II GP531, Shenzen GSD Tech Co., Ltd., China is used.

137 persons with type 1 diabetes were included, each of them used to intensive treatment with blood glucose self-monitoring 4-6 times per day, rapid mealtime insulin and long-acting insulin at bedtime or pump use. They were instructed in the use of the device used to measure non-invasively the glucose concentration using Raman Signal processing.

In addition the cohort of 23 patients with type 2 diabetes on oral antidiabetic drugs and/or insulin were under a similar test regimen as the group with type 1 diabetes. Twenty-six days of the study were used for calibration, followed by 15 days for validation. On each day patients performed 6 measurement units, each comprising 2 reference capillary tests and 3 NIGM scans in the sequence:
1. BGM reference
2. NIGM
3. NIGM
4. BGM reference, and lastly
5. NIGM.

Patients on insulin therapy controlled glucose and insulin dosing according to Self-monitoring Blood Glucose, but remained unaware of the NIGM readings. After instruction on the use of the NIGM device, there was no further professional supervision during the sessions for the days at home or work.

Capillary glucose, as standard for calibration and parallel measurement with NIGM, was measured with the Contour next One system (CNO, Ascensia, Switzerland). Accuracy in the hands of patients was found to correspond to a MARD of 5.6%

Control solution measurements were performed on the CNO test strips for every new strip vial opened before handing test strips to patients.

In FIGS. 7A to 7C, the dashed lines represent the average, subject-wise of the parameters in question over a fixed 15-day validation period, when the number of calibration days is varied between 4 and 26 days. The calculations include 160 subjects, where the grey band displays the 25-75% quantile range of subject-wise RMSE values. With nominally six measurement units per calibration day, it is important to note that a reduction in number of calibration days correspondingly reduces the size of the calibration set. It can be seen (FIG. 7C) that with a calibration period of 10 days it is still possible to achieve approximately 95% in the A+B zone.

FIG. 8 shows a comparison between the daily mean of the predicted and reference glucose value and RMSE for 160 patients for a validation period of 15 days. As can be seen there is very close correlation between the predicted and reference glucose values over the entire 15 day validation period. This is represented by a low value (<1.9) of RMSE for the entire validation window, albeit increasing slightly to approximately 1.84 at the end of the 15 day validation period (from approximately 1.68 at the start of the validation period).

Thus, an extended period of calibration is identified as a necessary feature to ensure stable calibration. Preferably, the period of calibration is at least 10 days, more preferably at least 14 days and more preferably still between 20 and 30 days. In the 3 specific examples demonstrated, an extended period of calibration stability is achieved using calibration periods of 14, 20 and 26 days. Furthermore, it follows that an extended calibration period, meaning a period of at least 10 days will in general provide calibration stability. Accordingly, whilst specific periods of 10, 14, 20 and 26 days are shown, it will be expected and understood that periods longer than this will also work. For example, periods of, say, 30 or 40, or more days would also be expected to provide the desired calibration stability.

Embodiments of the present invention have been described with particular reference to the examples illustrated. However, it will be appreciated that variations and modifications may be made to the examples described within the scope of the present invention.

1. Method of generating a stable calibrated model for use in a device for non-invasive in vivo measurement of an analyte concentration using Raman spectroscopy, the device comprising an optical source for providing an optical signal and an optical detector to receive a Raman scattered optical output, the method comprising:
in response to the laser source being directed to a user's skin, gathering Raman scattered radiation from the user's skin and based on that, calibrating the model based on reference data and the received Raman spectra over an extended period of days; and,
once calibrated after the extended period of days, storing the stable model for use.

2. The method according to claim 1, wherein the calibration period is at least 10 days.

3. The method according to claim 1, wherein the calibration period is at least 14 days.

4. The method according to claim 1, wherein the calibration period is at least 20 days.

5. The method according to claim 1, wherein the calibration period is between 20 and 28 days.

6. The method according to claim 1, comprising providing a model for calibrating and then modifying the model in dependence on calibration data received during the calibration period.

7. The method according to claim 1, comprising receiving reference data from an invasive blood glucose monitoring system or device.

8. The method according to claim 1 comprising releasing the calibrated model for use only if release criteria are satisfied.

9. A system for generating a stable calibrated model used in a device for non-invasive in vivo measurement of an analyte concentration using Raman spectroscopy, the device comprising an optical source for providing an optical signal and an optical detector to receive a Raman scattered optical output, the system comprising:
a laser source to direct laser light to a user's skin
a detector to gather Raman scattered radiation from the user's skin and
a processor arranged, based on the received Raman scattered radiation and on reference data, to calibrate the model over an extended period of days and,
once calibrated after the extended period of days, to store the stable model for use.

10. The system according to claim 9, wherein the calibration period is at least 10 days.

11. The system according to claim 9, wherein the calibration period is at least 14 days.

12. The system according to claim 9, wherein the calibration period is at least 20 days.

13. The system according to claim 9, wherein the calibration period is between 20 and 28 days.

14. The system according to claim 9, comprising providing a model for calibrating and then modifying the model in dependence on calibration data received during the calibration period.

15. The system according to claim 9, comprising receiving reference data from an invasive blood glucose monitoring system or device.

16. The system according to claim 9, comprising releasing the calibrated model for use only if release criteria are satisfied.

17. The method according to claim 1, comprising testing the model for stability by determining RMSE for the model and identifying the model as stable if the RMSE is less than 1.9 for at least a 15 day validation window.

18. The system according to claim 9, comprising testing the model for stability by determining RMSE for the model and identifying the model as stable if the RMSE is less than 1.9 for at least a 15 day validation window.

19. The method according to claim 1, comprising testing the model for stability by determining RMSE for the model and identifying the model as stable if the increase in RMSE over at least a 14-day validation period is less than or equal to 10%.

20. The system according to claim 9, comprising testing the model for stability by determining RMSE for the model and identifying the model as stable if the increase in RMSE over at least a 14-day validation period is less than or equal to 10%.
